(19) United States
(12) Patent Application Publication
(10) Pub. No.: US 2017/0248398 A1
(43) Pub. Date: Aug. 31, 2017

(54) FILM AND MIRROR INCLUDING CALIBRATED MARKINGS FOR REFERENCING BODY POSITION AND MOVEMENT

(71) Applicant: Sandee Cohen, New York, NY (US)

(72) Inventor: Sandee Cohen, New York, NY (US)

(21) Appl. No.: 15/054,334

(22) Filed: Feb. 26, 2016

(51) Int. Cl. G01B 3/00; A61B 5/107; A61B 5/11; A61B 90/00

(52) U.S. Cl. CPC G01B 3/00; A61B 90/39; A61B 5/1075; A61B 5/11; A61B 2090/3937

(57) ABSTRACT

A product comprising a film proportioned to fit on a surface of a mirror and adapted to electrostatically adhere to the surface of the mirror; the film comprising calibrated markings calibrated to allow a subject to accurately reference body position and body movements in the mirror when the film is adhered to the surface of the mirror. A mirror comprising calibrated markings including measurement demarcations and calibrated to allow a subject to accurately reference and measure body movements.

### BACKGROUND

Physical therapy is most effective with proper position, form and movement, for example maintaining a straight posture, shoulders, arms, hands, knees, etc. With an ordinary mirror, a patient has to try to find a background object to “spot” their progress. Patients also have to guess at body position, for example, guessing or “eyeballing” if their shoulders or arms are straight.

Patients and their care providers and trainers also attempt to assess rehabilitation exercises during sessions and longer periods of time to aid and assess progress. Also, patients often have to perform exercises many times a day over weeks or months to gain the best recovery, and may often find themselves having to perform exercises in different locations (e.g., at home, at work, at the doctor's office or gym).

### SUMMARY

Described is product comprising: a film proportioned to fit on a surface of a mirror and adapted to adhere to the surface of the mirror, the film comprising calibrated markings calibrated to allow a person to accurately reference body position and body movements in the mirror when the film is adhered to the surface of the mirror.

In embodiments the calibrated markings comprise a grid of regularly spaced horizontal lines and vertical lines. In embodiments the calibrated markings comprise regularly spaced dots or marks. In embodiments the calibrated markings comprise measurement demarcations. In embodiments, the measurement demarcations include symbols or alphanumeric measurements or both. In embodiments the measurement demarcations include a central x-axis and central y-axis. In embodiments the calibrated markings comprise a visibly distinguishable feature. In embodiments, the visibly distinguishable feature comprises a feature selected from the group of texture, shade, color, and reflectivity. In embodiments, the film is proportioned to fit at least a full-body mirror. In embodiments the film is adapted to be rolled-up. In embodiments the film is less than the size of a full-body mirror. In embodiments the film is configured to be aligned with another film on a surface of the mirror such that the calibrated markings align to allow the person to accurately reference body position and bodily movements in the mirror when the plurality of films is adhered to the mirror. In embodiments the film is configured to be aligned with another film on the surface of the mirror such that the calibrated markings align vertically or horizontally or both. In embodiments the calibrated markings include measurement demarcations, and the measurement demarcations align when the plurality of films is adhered to the mirror. In embodiments the film is configured to electrostatically adhere to the mirror. In embodiments the product further comprises a guide including one or more outlines for cutting or scoring the film in a shape of the outline.

In embodiments, described is a mirror comprising calibrated markings including measurement demarcations and calibrated to allow a person to accurately reference body position and measure body movements. In embodiments, the calibrated markings comprise a grid of regularly spaced horizontal lines and vertical lines. In embodiments the measurement demarcations include symbols or alphanumeric measurements or both.

In embodiments, described is a product comprising: a film proportioned to fit on a surface of a mirror and adapted to electrostatically adhere to the surface of the mirror, the film comprising calibrated markings including measurement demarcations and calibrated to allow a person to accurately reference body position and body movements when the film is adhered to the surface of the mirror.

### BRIEF DESCRIPTION OF THE DRAWINGS

FIGS. 1A-1B show an embodiment of a film including calibrated markings.

FIG. 2 shows an embodiment of a mirror including the electrostatically attached film with dark calibrated markings.

FIG. 3 shows an embodiment of a mirror including the electrostatically attached film with light calibrated markings.

FIGS. 4A-4B show an embodiment of a plurality of films with calibrated markings in a panel configuration for a mirror area.

FIG. 5 shows and embodiment of a film together with a cutting guide.

### DETAILED DESCRIPTION OF EMBODIMENTS

It is to be understood that the figures and descriptions of the present invention have been simplified to illustrate and describe elements that are relevant for a clear understanding of the present invention, while eliminating, for purposes of clarity, many other elements which are conventional in this art. Those of ordinary skill in the art will recognize that other elements are desirable for implementing the present invention. However, where such elements are well known in the art, and where they do not facilitate a better understanding of the present invention, a discussion of such elements is not provided herein. The present invention will now be described in detail on the basis of exemplary embodiments.

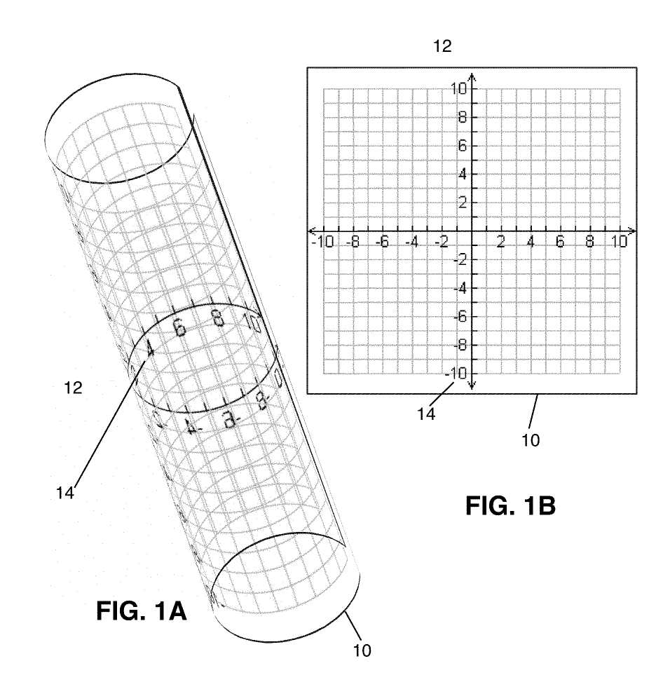

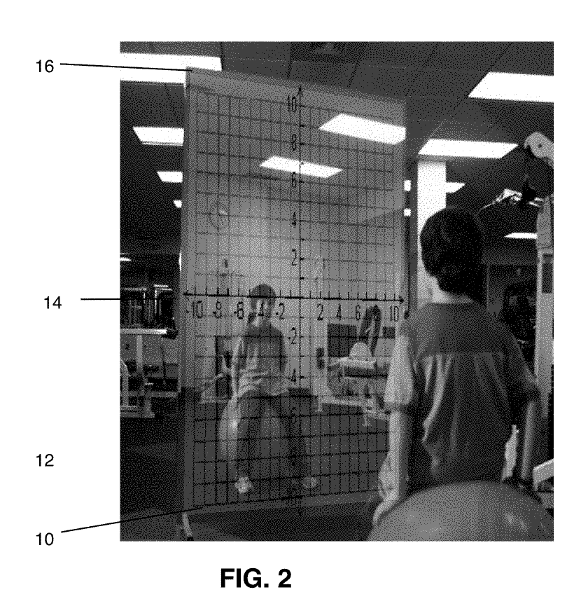

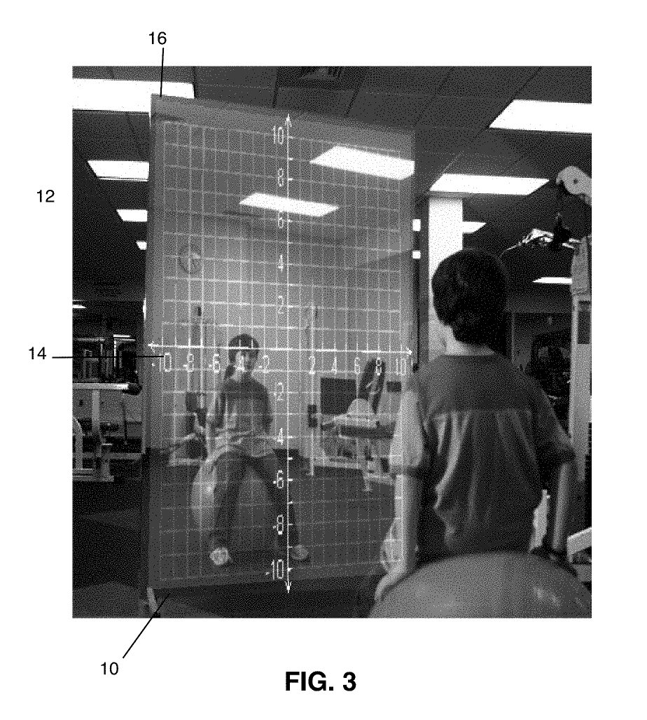

In at least one embodiment as shown in FIGS. 1A-3 is a product comprising a film 10 proportioned to fit on a mirror 16 and adapted to adhere to the mirror 16; the film 10 comprises calibrated markings 12 calibrated to allow a subject, for example a physical therapy patient, to accurately reference body position and body movements (e.g., recovery exercise movements) in the mirror when the film is adhered to the mirror. In an embodiment, the film can be an electrostatic film that electrostatically adheres to the mirror surface 16. As shown in FIG. 1B, when not in use the film 10 can be rolled up. The calibrated markings 12 can be printed on the film 10 or otherwise supplied on the film 10, for example as plastic or rubber markings added to the film. As shown in FIGS. 1A-3, the calibrated markings 12 can include a grid of regularly spaced horizontal lines and vertical lines supplied on the grid. In another embodiment (not shown) the calibrated markings 12 can comprise regularly spaced dots or marks. As shown in FIGS. 2 and 3, a person performing and/or monitoring physical therapy exercises or other bodily exercises in the mirror 16 can use the calibrated markings 12 to accurately reference body position and bodily movements when the film 10 is adhered to the mirror surface 16.

As shown in FIGS. 1A-3 the product can comprise the calibrated markings 12 comprising measurement demarcations 14. For example, as shown in FIGS. 1B-3 the grid on the film 10 includes measurement demarcations 14 including a central bolded x-axis and central bolded y-axis with alphanumeric measurements and hashes, shown as numeric coordinates on the central x-axis and y-axis. The measurement demarcations 14 allow a person measure and plot body movements during exercise, for example to measure improvements and milestones in recovery from an injury or other physical goals. In embodiments the measurement demarcations 14 can be symbols or alphanumeric measurements indicating a specific measure (e.g., feet, inches, meters, centimeters, etc.).

In at least one embodiment, the calibrated markings 12 can comprise one or more visibly distinguishable features. Visibly distinguishable calibrated markings 12 can be employed for various advantages, for instance, to improve the visibility of the markings on the mirror when the mirror is placed in contrasting surroundings that reflected in the mirror. For example, in an embodiment as shown in FIG. 2, the film is supplied with dark or calibrated markings 12, shown as black grid lines and black bolded measurement demarcations 14, shown as the x-axis and y-axis and numbers in black bold. In another embodiment, as shown in FIG. 3, the film is supplied with light calibrated markings 12, shown as white grid lines and white bolded measurement demarcations 14, shown as the x-axis and y-axis and numbers in white bold.

As will be appreciated, other colors and color combinations can be employed. For example, calibrated markings 12 can be offered in different colors or even customized for specific environments, for example to coordinate with interior design or branding. Different color combinations can be employed to, for example, have the measurement demarcations 14 contrast with the other calibrated markings 12. In embodiments, the visibly distinguishable calibrated markings 12 can include other visibly distinctive features, for example, texture or adjusted reflectivity. For example, calibrated markings 12 can include or be made from plastic or rubber that is visibly raised from the film 10. In another embodiment, the reflectivity of the calibrated markings 12 can be adjusted, for example, employed a matte textured material (e.g. roughened plastic or rubber) to absorb light to further contrast the markings when the film 10 is adhered to the mirror 16.

In an embodiment, the film 10 is designed and proportioned to fit and substantially overlay a full-body mirror 16 or larger. For example, physical therapy facilities often use 7 ft.×20 ft. mirrors; hence in an embodiment a film 10 can be about 6½ ft.×18½ ft. so as to fit and substantially overlay the mirror surface 16 when applied to the mirror 16. In embodiments, the film 10 can be proportioned to fit other mirrors 16, such as dressing mirrors or bathroom mirrors commonly found in the home or workplace.

In another embodiment, the film 10 can be less than full body sized but large enough to allow a user to reference and measure body or body part movements. For example, in an embodiment the film 10 can be configured to be size that is easily portable, for example in a range between about 3 ft.×3 ft. to about 6 ft.×5 ft.

As will be appreciated the film 10 can be made other sizes to fit within and/or overlay different-sized mirror 16 surfaces. In embodiments, the film 10 can be proportioned to be a size to cover full body exercises independent of mirror 16 size. For example a film 10 can be a square 6½ ft.×6½ ft., which while proportioned to fit within most mirrors 16 for most commercial exercise venues, for example physical therapy offices or gyms, the film 10 is not proportioned specifically to fit a particular mirror 16 size. In another embodiment, the film 10 less can be than full body sized, but can include regular calibrated markings 12 to allow them to be stacked or paneled to cover full body exercises or in other configurations (e.g. to fit a particular mirror). In embodiments the films 10 can be different shapes, for example circular, oval, rectangular, polygonal, or even whimsical or specially designed shapes, for example character outlines or animal outlines. For example, a film 10 can be a large film outlined in the shape of a readily recognizable figure, such as a strongman, strongwoman, monster, or a famous fictional character (e.g., Big Bird®, the Hulk®, Ms. Marvel®), and the inside of the outline of the film 10 includes calibrated markings 12 and measurement demarcations 14 such as a grid as described herein.

In an embodiment the film 10 can be adapted to be cut to a size and/or shape using a cutting or scoring device. For example, in an embodiment the calibrated markings 12 or the calibrated markings 12 including the measurement demarcations 14 can be used to gauge or measure the interior surface of the film 10, allowing a user to cut the film 10 to fit a given mirror 16 size or otherwise cut the film 10 to a desired size using scissors or a knife or other cutter. For instance, in an embodiment where the calibrated markings 12 include a grid as described herein, the grid lines can be employed to cut the outer portions of a film 10 to a desired size, for example to fit on the surface of a smaller mirror 16. In embodiments where the calibrated markings 12 include measurement demarcations 14, for example symbols or alphanumeric numbers indicating a specific measure (e.g., feet, inches, meters, centimeters, etc.), the measurements on the film 10 can be used to cut the film to size.

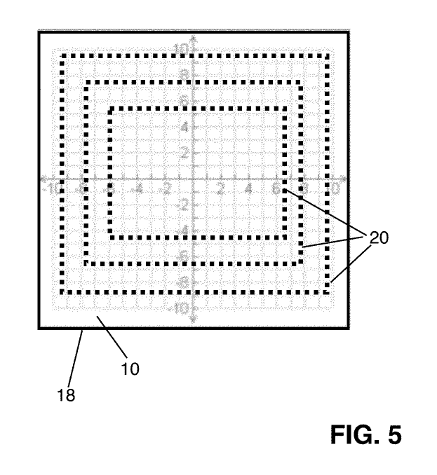

In another embodiment, a film 10 can be provided with a separate guide 18 to cut the film 10 to a size and shape. For example, as shown in FIG. 5, the film 10 could be provided with a guide 18 comprising a sheet to be placed under the film 10 or over the film 10, the sheet including one or more predefined shape outlines 20, which can be used to guide a user when cutting the film 10. The film 10 can then be cut or scored over the one of the guide 18 outlines 12. In embodiments, the guide 18 can be made of a material that can also be cut, paper or light cardboard for example, and the film 10 and the guide 18 can be cut together along the outline 20.

In various embodiments the film 10 includes materials to be removably mountable to a smooth reflective surface such as a mirror. In an embodiment the film 10 is removably mountable using static cling or cohesive forces as described above. For example some or all of the film can include or can be formed of plasticized polyvinylchloride (PVC) film or electrostatically-charged oriented polypropylene (PP) film.

While static or cohesive cling are described herein as the force by which the film 10 is mounted to the mirror, it should be understood that other removable mounting methods and elements are amenable to use with the various embodiments described herein, and are within the scope of the disclosure. For example, thermoplastic adhesives may be applied to the film 10 to provide the necessary cling. In another embodiment, mounting elements can be added to the film or supplied with the film to removably adhere the film to the mirror. For example hook-and-loop mounts can be added to the corners and sides of the film 10 and mating mounts applied a mirror to allow the film to be repeatedly adhered and removed from the mirror using the hook-and-loop mounts.

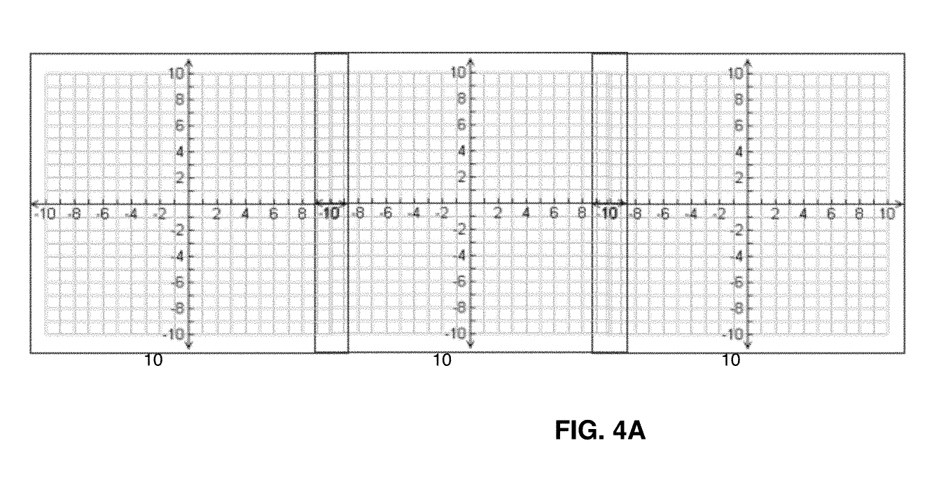

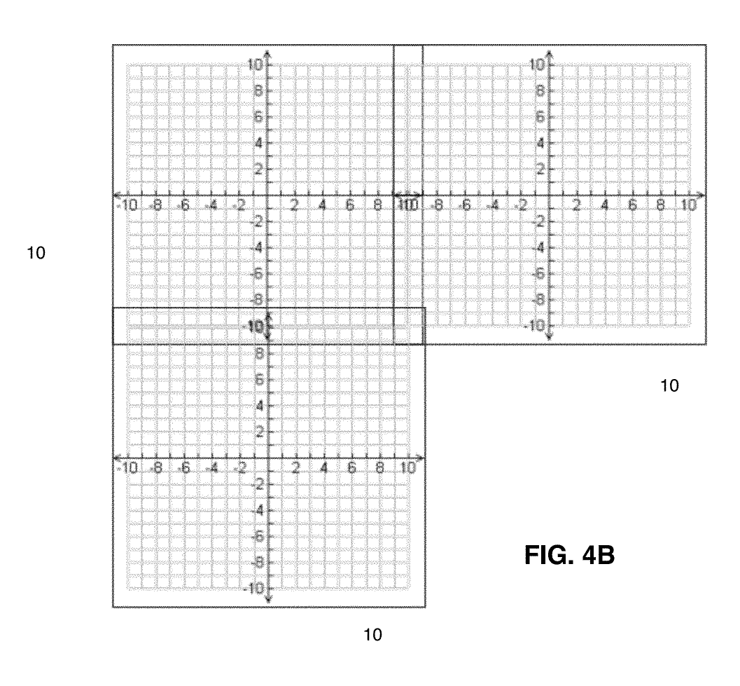

In another embodiment, the film 10 can be configured to be placed with other films 10 to cover mirror areas, for example to be employed in environments with large mirrors, unconventionally shaped mirrors, multiple mirrors, or mirror panels. For example, with a film 10 including a grid as shown in FIGS. 1A-1B, the x-axis of multiple films can be aligned as the grids are placed side-by-side horizontally as shown in FIGS. 4A and 4B, and the y-axis of multiple films can be aligned when stacked vertically as shown in FIG. 4B. As shown in FIGS. 4A-4B, the measurement demarcations 14 are in sets of ten (10) so as to facilitate plotting when the grids are aligned.

In an embodiment, a method for using the film 10 comprises unrolling the film, placing and aligning the film within a mirror surface area and adhering the film to the mirror such that it is fitted to and/or in the mirror surface. In embodiments, multiple films 10 can be unrolled and/or aligned with the mirror and with one another, for example to panel a mirror as shown in FIGS. 4A-4B. In embodiments, the film 10 can be aligned to the mirror in a location that is optimal for the subject doing the exercises. For example, in the case of a small child or pet, a smaller film 10 (e.g. from about 3 ft.×3 ft. to about 5 ft.×5 ft.) may be positioned lower on the mirror such that the calibrated markings 12 and measurement demarcations 14 are centered on the smaller frame of the subject.

In another embodiment, described is a mirror 16 comprising calibrated markings 12 including measurement demarcations 14 and calibrated to allow a subject to accurately reference body position and bodily movements, for example a physical therapy patent referencing recovery exercise movements. The calibrated markings 12 can be the same as those described on the films above, including with respect to FIGS. 1A-3, for example a grid of regularly spaced horizontal lines and vertical lines supplied directly on the mirror 16 along with measurement demarcations 14 such as numbered and bolded central x-y axes. In another embodiment (not shown) the calibrated markings 12 comprise regularly spaced dots or marks. The calibrated markings 12 can comprise one or more visibly distinguishable features as described above. The calibrated markings can be printed, etched, laminated, deposited or otherwise added to the mirror surface 16.

In an embodiment, a method of using a mirror with calibrated markings and/or measurement demarcations is described. A subject stands in front of the mirror and performs a particular bodily movement or set of movements, for example a patient recovering from an injury performs rehabilitation exercises. While doing the exercise, the subject or a facilitator (e.g. doctor, physical therapist, trainer) uses the grid lines to reference and judge body position. For example, a patient, doctor, physical therapist or trainer can assess and reference whether the subject is standing up straight, or whether a particular body parts are in alignment. The measurement demarcations 14 can then be used to assess the exercise, for example to measure or plot any improvement or degradation in movement, milestones, range of motion, etc. As will be appreciated, the calibrated markings 12 can be applied to the mirror 16 with the film 10 or multiple films 10 or can calibrated markings 12 be printed or supplied directly on the mirror 16 as described in the embodiments herein.

Although embodiments have been described in terms of physical rehabilitation of a patient, embodiments can be employed for any activity where referencing, measuring and tracking body movement in a mirror is desirable. For example, embodiments can be used for sports and athletic training, fitness where proper body position is desirable (weight-training, yoga), dance, and so on.

Although specific embodiments have been illustrated and described herein, it will be appreciated by those of ordinary skill in the art that other arrangements can be substituted for the specific embodiments shown. This application is intended to cover any adaptations or variations of the present invention. Therefore, it is manifestly intended that this invention be limited only by the claims and the equivalents thereof.

What is claimed is:

1. A product comprising:
a film proportioned to fit on a mirror surface and adapted to adhere to the surface of the mirror,
the film comprising calibrated markings calibrated to allow a person to accurately reference body position and body movements in the mirror when the film is adhered to the surface of the mirror.

2. The product of claim 1 further comprising:
the calibrated markings comprising a grid of regularly spaced horizontal lines and vertical lines.

3. The product of claim 1 further comprising:
the calibrated markings comprising regularly spaced dots or marks.

4. The product of claim 1 further comprising:
the calibrated markings comprising measurement demarcations.

5. The product of claim 1 wherein measurement demarcations include symbols or alphanumeric measurements or both.

6. The product of claim 2 wherein the measurement demarcations include a central x-axis and central y-axis.

7. The product of claim 1 wherein the product further comprises:
a guide including one or more outlines for cutting or scoring the film in a shape of the outline.

8. The product of claim 1 wherein the calibrated markings comprise a visibly distinguishable feature comprising a feature selected from the group of texture, shade, color, and reflectivity.

9. The product of claim 1 wherein the film is proportioned to fit and substantially overlay at least a full-body mirror.

10. The product of claim 1 wherein the film is from about 3 ft.×3 ft. to about 7 ft.×20 ft..

11. The product of claim 1 wherein the film is adapted to be rolled-up.

12. The product of claim 1 wherein the film is less than the size of a full-body mirror.

13. The product of claim 1 wherein the film is configured to be aligned with another film on a surface of the mirror such that the calibrated markings align to allow the person to accurately reference body position and bodily movements in the mirror when the plurality of films is adhered to the mirror.

14. The product of claim 13 wherein the film is configured to be aligned with another film on the surface of the mirror such that the calibrated markings align vertically or horizontally or both.

15. The product of claim 13 wherein the calibrated markings include measurement demarcations, and the measurement demarcations align when the plurality of films is adhered to the mirror.

16. The product of claim 15 wherein the film is configured to electrostatically adhere to the mirror.

17. A mirror comprising calibrated markings including measurement demarcations and calibrated to allow a person to accurately reference body position and measure bodily movements.

18. The mirror of claim 17 further comprising:
the calibrated markings comprising a grid of regularly spaced horizontal lines and vertical lines.

19. The mirror of claim 17 wherein measurement demarcations include symbols alphanumeric measurements or both.

20. A product comprising:
a film proportioned to fit on a surface of a mirror and adapted to electrostatically adhere to the surface of the mirror;
the film comprising calibrated markings including measurement demarcations and calibrated to allow a person to accurately reference body position and body movements when the film is adhered to the surface of the mirror.
